(19) United States
(12) Patent Application Publication
(10) Pub. No.: US 2025/0154491 A1
(43) Pub. Date: May 15, 2025

(54) PROCESS OF OBTAINING A PROTEIN PREPARATION

(71) Applicant: INSTITUTO SUPERIOR DE AGRONOMIA, Lisboa (PT)

(72) Inventors: Ricardo Manuel DE SEIXAS BOAVIDA FERREIRA, Lisboa (PT); Madalena DE MATOS ÁGUAS GRÁCIO, Odivelas (PT); Giovanni DEL FRARI, Lestans (IT)

(73) Assignee: INSTITUTO SUPERIOR DE AGRONOMIA [PT/PT], Lisboa (PT)

(21) Appl. No.: 18/837,593

(22) PCT Filed: Feb. 9, 2023
(86) PCT No.: PCT/EP2023/053261

§ 371 Date: Aug. 10, 2024

(30) Foreign Application Priority Data
Feb. 10, 2022 (GB) 2201737.0
Feb. 10, 2022 (PT) 117785

(51) Int. Cl. C12N 9/88

(52) U.S. Cl. CPC C12N 9/88; C12Y 401/01039

(57) ABSTRACT

A process of obtaining a protein preparation from biological tissues selected from the group comprising seeds eg. legume seeds, algae eg. macro or microalgae, bacteria eg. cyanobacteria, animal products, said process comprises the steps of:
lysing said biological tissues to extract protein; and
submitting at least a portion of said extracted material to an activated charcoal; whereby a portion is removed by or adsorbed onto said activated charcoal; and/or submitting a portion of extracted material to a solution containing EDTA eg. of 10 to 40 mM; and/or submitting a portion of extracted material to a solution containing NaCl eg. of 5 to 15% (w/v); and/or submitting a portion of extracted material to a bentonite concentration; and/or submitting a portion of extracted material to a solution containing phytate eg. of 7.5 to 30 mM; and/or submitting a portion of extracted material to a phosphate eg. of 25 to 100 mM; whereby one or more of the following proteins are isolated: legume seed proteins, RuBisCO, soluble protein from leafy vegetables either edible or non-edible, macro or micro algae protein, cyanobacteria protein, animal products protein.

### FIELD OF THE INVENTION

Aspects of the invention relate to the process for obtaining a protein preparation. In particular, aspects of the invention improve the extraction process for one or more of the following proteins: legume seed proteins, RuBisCO, soluble protein from leafy vegetables either edible or non-edible, macro or micro algae protein, cyanobacteria protein, animal products protein.

In particular aspects of the invention relate to the process for obtaining a RuBisCO preparation from a photosynthetic material.

RuBisCO is to be interpreted broadly and may include at least the following definitions: ribulose-bisphosphate carboxylase, RuBP carboxylase, diphosphoribulose carboxylase, ribulose 1,5-diphosphate carboxylase, carboxydismutase, ribulose 1,5-biphosphate carboxylase and ribulose 1,5-di(or bis) phosphate carboxylase-oxygenase, 3-phospho-D-glycerate carboxy-lyase (dimerizing; D-ribulose 1,5-bisphosphate-forming); 3-phospho-D-glycerate carboxy-lyase (dimerizing).

Preferred aspects concern the large scale purification of a protein for human and animal consumption.

### BACKGROUND TO THE INVENTION

The worldwide need for sustainable protein production for human and animal feed has never been greater. In addition, there is also an increasing demand for plant derived protein. Many attempts have focused on developing a large-scale strategy capable of purifying at a reasonable price the most abundant protein in nature, RuBisCO (E.C. 4.1.1.39): the enzyme catalyzing the first reactions of two metabolically opposed pathways, the Calvin cycle and photorespiration. Each molecule of plant RuBisCO is a hexadecamer composed of eight large subunits (LSU; ca. 475 amino acid residues or 50 to 55 kDa each) and eight small subunits (SSU; ca. 125 amino acid residues or 12 to 15 kDa each). It is therefore a very large protein, composed of approximately 4,800 amino acid residues and a molecular mass of about 550 kDa. RuBisCO is recognized as a sluggish catalyst with a turnover number around 15 CO₂ molecules fixed per s per enzyme molecule, a number which drops to ca. 3 under optimum agricultural conditions—compare to catalase, where a single enzyme molecule can decompose many millions of hydrogen peroxide molecules per second under optimal conditions. To cope with the unavoidable extremely low RuBisCO catalytic efficiency and with the fact that the enzyme spends, for the vast majority of crops and depending on the environmental conditions, between 25 and virtually 100% of the time fixing oxygen in photorespiration rather than carbon dioxide in the Calvin cycle, plants accumulate massive amounts of RuBisCO in their photosynthetic tissues in order to grow at reasonable rates. For the vast majority of crops, this means that a single protein (i.e., RuBisCO) comprises typically over 50% of the total leaf proteins, where we may find over 10,000 other different proteins. In other words, RuBisCO comprises ca. 50% of the total leaf protein in almost all the plants consumed, meaning that it is ca. 50% pure in the leafy vegetables that are eaten.

Immediate consequences derived from the above comments are that everyone eating photosynthetic tissues ingests large amounts of RuBisCO. The nutritional quality of RuBisCO as an edible protein is unsurmountable:
It is a natural compound, present in very high amounts in all green plant tissues consumed;
It is non-toxic when ingested in any amount;
It is readily digested by the proteases of our digestive system;
It is non-allergenic; indeed, it is often used as a negative control in allergenicity tests;
From the human nutritional point of view, it is the commercially available protein closer to the ideal protein, as defined by FAO (Food and Agriculture Organization of the United Nations) (chemical index=100 when compared to the ideal protein recommended by FAO/WHO, 2007); thus, among animal proteins, egg protein is far too rich in what the branched-chain amino acids are concerned (eggs are also a source of allergens), whereas some whey proteins are potent allergens and expensive when commercialized in the pure form; among plant proteins, legume seed proteins (e.g. soybean, pea, etc.) are also potent allergens and rather poor in the sulfur-containing amino acids (cysteine and methionine), whereas cereal proteins are deficient in lysine. RuBisCO is a well-balanced protein, with a proportion of essential amino acids that equals or exceeds that recommended by FAO. Each RuBisCO molecule contains almost 200 sulfur atoms, ca. 100 derived from cysteine and ca. 100 from methionine residues.

It is believed that RuBisCO large-scale extraction and purification for further application in the food industry is a process that could solve several global problems such as protein malnutrition, a well-known public health problem (Müller & Krawinkel, 2005; Stefano et al., 2018).

Virtually all proteins which are commercially available for human and animal consumption are integral members of the lists of the major allergens both in Europe and the US—but not RuBisCO. Based on all its well-studied characteristics, it seems clearly established that RuBisCO may be regarded as the ideal protein for both human and animal consumption. It is abundantly present in all photosynthetic tissues and is often used as a negative control in allergenicity studies. With all the information provided above, it is somewhat strange that up to this moment no one has yet developed a methodology for the non-expensive, large scale RuBisCO purification in the form of a colourless, odourless and tasteless soluble purified powder, yielding RuBisCO that may be considered, simultaneously, as organic, generally regarded as safe and obtained by a clean extraction procedure. Many procedures have been described in the literature, but they are either non-scalable due to the complexity of the purification protocol or to the huge price of the resulting pure product, or produce impure, insoluble, unpleasantly tasting and/or green preparations of RuBisCO.

Aspects of the invention provide a sustainable and scalable process for the purification of RuBisCO from photosynthetic cells/tissues/organs/entire organisms. In certain embodiments, the final product, RuBisCO, exhibits a high degree of purity and yield, and consists of a white powder, devoid of pigments (e.g., chlorophylls and carotenoids), obtained without the use of organic solvents or any other toxic compound, with no odour, smell or taste, something which has not reached the market yet.

### PRIOR ART KNOWN TO THE APPLICANTS

### Existing Methods for Large Scale Purification of RuBisCO

There are several methods of purifying RuBisCO in the laboratory, which are not amenable to adaptation for large scale purification at an industrial level and a reasonable price. This explains why, to date, no pure RuBisCO is on the market, with a multitude of potential applications. However, although there are some methods in published patent databases that apparently allow for large-scale purifications, in fact they only extract the total soluble protein or produce highly impure or insoluble and/or green-coloured RuBisCO, and there is still no simple and economical method to isolate large amounts of the pure enzyme (in the order of kg and more) based on a methodology that is compatible with the food industry.

In 1981, a crystallization method with the toxic 2-mercaptoethanol was described in U.S. Pat. No. 4,268,632, in which a purification of 90% of RuBisCO was obtained. This method requires temperature control, since protein extraction is carried out by heating to 50° C. (non-denaturing temperature for RuBisCO), and subsequent cooling to temperatures lower than room temperature, to allow adequate formation of protein crystals.

In 1982, another large-scale purification method was published in U.S. Pat. No. 4,400,471, through crystallization with PEG (polyethylene glycol). This method uses different amounts of PEG, between 8 and 13% (w/v), under controlled pH and temperature, thus allowing a purification equivalent to the method described in the previous paragraph. In the same patent publication, it is also mentioned the use of an ion exchange resin, which allows a considerable degree of purification. The author states that RuBisCO is the only compound that can bind to this resin, so its use allows a good degree of purification, after dissociation of the resin-RuBisCO complex using a solution of divalent metal ions. Alternatively, the two methods can also be combined, first crystallizing and then passing RuBisCO through the resin, thus achieving an even higher degree of purification (>90%).

The purification methods most often used to obtain RuBisCO for rheological studies involve precipitation at pH 3.5 of the soluble extract of alfalfa (Medicago sativa), in which a precipitation of almost all proteins is obtained by denaturation, the majority of which is RuBisCO, precipitation with ammonium sulfate and an affinity separation of spinach leaf soluble extract (WO2011078671).

Tenorio et al. in 2017 used sugar beet for the purification of proteins from their leaves, considered as a waste byproduct. The proteins were extracted by applying a precipitation at high temperatures (50° C.). The heating of proteins can cause their denaturation and should therefore be a step to be avoided. As RuBisCO is found in leaves in large quantities, it is reported that this methodology is capable of purifying RuBisCO up to 90% of the total protein—This may be an unexpected result, as RuBisCO has been claimed not to denature at 50° C. On the other hand, the final product is expected to comprise the total soluble leaf proteins existing in the beet leaf, probably enriched in those proteins which tend to precipitate at 50° C.

In summary, the methods that have been developed for RuBisCO purification show several disadvantages which make them inappropriate for the large scale purification required for the commercial availability of pure RuBisCO, such as: the purification procedures, which usually comprise very extensive protocols, with a high number of steps and most often unsuitable to undergo scaling-up; use of organic solvents that may cause denaturation/aggregation, decrease the solubility of RuBisCO and leave residues harmful to human health, leading to a final product that cannot be considered as (i) organic, (ii) generally regarded as safe and (iii) obtained by a clean extraction procedure (using exclusively water and ethanol as solvents); and the presence of chlorophylls, which gives a green colour to the final product.

In recent years, some companies have claimed their success in isolating this protein, although it has been found that they have only obtained a protein concentrate, where RuBisCO is the major protein. These extracts may contain compounds that will contribute negatively to their quality, namely the presence of potentially allergenic proteins, phenolic acids and tannins, as well as residues from the purification procedure itself, which may be harmful to human health.

### RuBisCO-Producing Companies

There are currently companies that claim to purify RuBisCO on a large scale, namely France Luzerne, TNO and NIZO. However, the first obtains only a total soluble extract with a low degree of RuBisCO purification. It is worth remembering that RuBisCO is already ca. 50% pure (relative to the total leaf protein) in the leaves of C3 plants. This extract may contain potentially allergenic and/or other undesirable proteins, tannins and other phenolic compounds, which can negatively contribute to the nutritional quality of the protein concentrate.

The first company that produced RuBisCO on a large scale was France Luzerne, a French company participating in Fralupro, a European project whose objective was to discover a possible use for 32 million ha of alfalfa. In 1998, this company started to produce 1,200 tons of alfalfa RuBisCO per year, at the same cost of producing soy protein. The purified RuBisCO, with a low degree of purification, is used in animal feed (rations) and in several areas of human nutrition—e.g., cookies.

TNO—Innovation For Life is a Dutch company that also produces RuBisCO on a large scale, from sugar beet leaves. The process involves pressing, centrifuging and ultrafiltration, and has a production capacity of 10 kg protein/h, with a low degree of purification, around 35 to 40%. However, as mentioned earlier, these two companies do not produce RuBisCO in a purified state, as it is contaminated with other proteins and compounds from the plant leaves where it derives from.

Another Dutch company, NIZO, filled its RuBisCO purification process as a patent application in 2010. This process consists of protein extraction, with decantation and pressing, followed by purification by aggregation, precipitation and affinity separation, with final concentration by filtration and evaporation. The final product contains a minimum of 90% of the soluble RuBisCO and less than 0.1% (w/v) chlorophylls. However, the process is complex and has not yet been able to be scaled to an industrial level.

RuBisCO Foods is a Dutch company that produces protein gels and powders for the purpose of their application in food and feed. This company has developed and patented a unique technology that allows the extraction and purification of existing proteins in Lemna spp. (aquatic flowering plants). This plant has a significant growth rate, having the ability to double its biomass every 36 h, under optimum conditions, suitable for a daily harvest. Its rapid growth allows the production of 7 times more protein per ha of soil than soybeans. Therefore, this plant not only grows rapidly but also due to the fact that the plant is used entirely, i.e., not leaving any residues or waste. Unfortunately, the products manufactured by this company, gels and powders, have a green color, which indicates that the final product includes chlorophylls in its composition. In addition, RuBisCO in the final product is not pure at all.

GreenProteins is a European project that presents as its main objective the production of high-quality proteins for later application in food. Proteins are extracted from sub-products produced by the food industry. This project, which began in 2016 and will end in 2021, was based on a new method for purifying RuBisCO from sugar beet leaves as described in US20150335043A1. Although the final product achieved is a white powder, which indicates the removal of chlorophylls, the described method uses organic solvents for the removal of chlorophylls, which is neither compatible with healthy food nor feasible for further application in the food industry. It is also important to note that, despite the fact that in the disclosure made to the public it is mentioned that RuBisCO is purified, the final product that is produced is the total extract of soluble proteins existing in the sugar beet leaves.

### SUMMARY OF THE INVENTION

In a broad aspect, the invention provides a process of obtaining a protein preparation from biological tissues selected from the group comprising seeds eg. legume seeds, algae eg. macro or microalgae, bacteria eg. cyanobacteria, animal products, said process comprises the steps of:
lysing said biological tissues to extract protein; and
submitting at least a portion of said extracted material to an activated charcoal; whereby a portion is removed by or adsorbed onto said activated charcoal; and/or submitting a portion of extracted material to a solution containing EDTA eg. of 10 to 40 mM; and/or submitting a portion of extracted material to a solution containing NaCl eg. of 2.5 to 15% (w/v); and/or submitting a portion of extracted material to a bentonite concentration; and/or submitting a portion of extracted material to a solution containing phytate eg. of 7.5 to 30 mM; and/or submitting a portion of extracted material to a phosphate eg. of 25 to 100 mM; whereby one or more of the following proteins are isolated: legume seed proteins, RuBisCO, soluble protein from leafy vegetables either edible or non-edible, macro or micro algae protein, cyanobacteria protein, animal products protein.

In a further broad aspect, the process of obtaining a RuBisCO preparation from photosynthetic material, comprises the steps of lysing said photosynthetic material to extract RuBisCO accompanied by a fraction of the chlorophylls of said photosynthetic material; submitting at least a portion of said extracted material to an activated charcoal; whereby a further portion is removed by or adsorbed onto said activated charcoal.

This process is particularly advantageous because in preferred embodiments it may revolutionize the supply of (essentially plant derived) proteins. In preferred embodiment, the isolated proteins may comprise the total soluble leaf, macroalgae, microalgae and cyanobacteria protein; the legume seed protein; and pure RuBisCO.

In preferred embodiment, the process provides for the exquisite precision application of activated charcoal to differentially adsorb distinct protein fractions, pigments and the metabolites responsible for the unpleasant taste.

Advantageously, in certain embodiments, the process is capable of removing residual amounts of undesirable compounds, such as pesticides, antibiotics, hormones and heavy metals, as well as some endogenous unwanted metabolites, such as the phytoestrogens isoflavones present in soybean seeds. In a preferred embodiment, the protein seed comprises protein from soybean seeds.

Advantageously, such proteins are extracted from edible and non-edible agricultural crops and other herbaceous and perennial plants (e.g. plant leaves); wastes from the agricultural and the agro-food industries (circular economy); macro- and microalgae, and cyanobacteria.

Legume seeds such as soybean, pea, lupin, chickpea, etc. and other seeds whilst for the first time being in a colourless and tasteless form. This procedure may be extended to isolate proteins from animal or other sources.

In a subsidiary aspect, said further portion comprises chlorophyll pigments.

In a subsidiary aspect, the process comprises a step of grinding leaves containing photosynthetic material and adding water to obtain an aqueous solution.

In a subsidiary aspect, the process further requires a step of centrifugation to separate out pellets and a supernatant comprising the soluble proteins which were not adsorbed onto the activated charcoal.

In a further subsidiary aspect, the process further comprises one or more further steps of nanofiltration, ultrafiltration or microfiltration; whereby soluble RuBisCO, total soluble leaf proteins, legume seed proteins may be isolated. Advantageously, these are obtained in the pure state as a white or whitish, odourless, and tasteless soluble powder.

This configuration is particularly advantageous as when activated charcoal is combined with micro, ultra- and nanofiltration, it allows further improvement to the efficient and differential fractionation and separation of coloured pigments, metabolites (including those responsible for unpleasant taste such as, for example, those present in legume seed, microalgae and cyanobacteria proteins currently commercially available), pesticide residues, antibiotic residues, hormone residues, and heavy metals from non-RuBisCO proteins and from RuBisCO. Consequently, a more extensive methodology allows for obtaining highly pure RuBisCO.

In preferred embodiments, the soluble RuBisCO is colourless, odourless and tasteless.

In preferred embodiments, the isolated protein is soluble.

In preferred embodiments, the isolated protein is free from pesticide, antibiotic and heavy metal residues, since both the initial incubation with activated charcoal and the final nanofiltration step combine to remove them. In further preferred embodiments, the isolated protein obtained by the process is free from other residues of toxic compounds, such as organic acids for example.

In a further subsidiary aspect, the process comprises a one-step method for the large-scale isolation of RuBisCO and the total soluble protein from plant leaves, from macro and microalgae, cyanobacteria and from agricultural and food industry wastes.

In a further subsidiary aspect, the process comprises the step of agitating and/or centrifuging the aqueous solution in order to obtain a pellet of insoluble particles and a supernatant.

In a further subsidiary aspect, the process comprises the step of adding the activated charcoal to the supernatant and further processing the solution whereby said activated charcoal adsorbs chlorophyll pigments.

In a further subsidiary aspect, the activated charcoal is added in a range of 0.25% to 2.5% (w/v).

In a further subsidiary aspect, said further processing results in a pellet of low molecular mass molecules, ions and/or atoms and a supernatant of soluble proteins which were non-adsorbed onto the activated charcoal.

In a further subsidiary aspect, the process further comprises a step of centrifugation to separate out pellets and a supernatant comprising the soluble proteins which were not adsorbed onto the activated charcoal.

In a further subsidiary aspect, the process further comprises one or more further steps of nanofiltration, ultrafiltration or microfiltration; whereby soluble RuBisCO is isolated.

In a further subsidiary aspect, the nano-filtration employs a PES (polyethersulfone) membrane of for example 0.22 μm.

In a further subsidiary aspect, the nano-filtration employs an ultra-filtration membrane with a molecular weight cut-off (MWCO) of for example 10 kDa to 100 kDa.

In a further subsidiary aspect, the step of filtration employs a molecular weight cut-off (MWCO) membrane of 100 kDa or less.

Aspects of the invention envisage step employing 0.22 m, 100 kDa and 10 kDa cut-off membranes, respectively. The 0.22 m (or similar) cut-off membrane will retain larger, insoluble particles such as chunks of unbroken cells, plant cell wall fragments, starch grains, intact organelles, hydrophobic protein aggregates, etc.; the 100 kDa (or similar) cut-off membrane will retain very large proteins (e.g., RuBisCO); the 10 kDa (or similar) cut-off membrane will retain the smaller proteins (the vast majority of the intracellular proteins and virtually all extracellular proteins), but not all the small molecular mass molecules, elements and ions, such as metabolites and salts, including traces of pesticides, antibiotics, hormones and heavy metals.

In a further aspect, the invention provides a RubisCO preparation obtained by a process according to any one of the preceding aspects.

Embodiments provides a high-yield and high-efficiency methodology based on the combined use of the exquisite precision application of activated charcoal to differentially adsorb distinct protein fractions, pigments and the metabolites responsible for the unpleasant taste; and/or micro-, ultra- and nanofiltration to produce isolated proteins in the form of a soluble, tasteless, odourless, colourless (white), free from small molecules (including natural toxins and residues of pesticides, antibiotics, hormones, and organic solvents) powder to obtain
RuBisCO:
total soluble protein from plant photosynthetic tissues (e.g., leaves)
total soluble protein from macro- and microalgae, and from cyanobacteria
total soluble protein (including the ‘solubilized’ storage proteins) from legume seeds.

Under some conditions, the total soluble protein isolated from certain cyanobacteria (e.g., Spirulina) may retain a small part of its initial green color.

In certain embodiments, the above procedure obtains pure RuBisCO and/or total soluble proteins in the form of a soluble, tasteless, odourless, colourless (white), free from small molecules (including natural toxins and residues of pesticides, antibiotics, hormones, and organic solvents) powder from:
The photosynthetic tissues (e.g., leaves) of herbaceous plant edible plants (e.g., spinach);
The photosynthetic tissues (e.g., leaves) of edible herbaceous plants, commonly originated by agricultural waste or agro-food industries:
radish/beetroot/turnip/carrot/broccoli/sweet potato/squash/pumpkin/cucumber/bell pepper/tomato/eggplant/beans—the leaves of common green beans (Phaseolus vulgaris), yardlong beans (Vigna unguiculata), runner beans (Phaseolus coccineus), lima beans (Phaseolus lunatus), fava beans (Vicia faba), and hyacinth beans (Lablab purpureus) are 100 percent edible;
Edible aquatic plants (e.g., Lemna minor);
The photosynthetic tissues (e.g., leaves) of non-edible herbaceous plants, commonly originated by agricultural waste or agro-food industries (e.g., legume crops, potato);
The photosynthetic tissues (e.g., leaves) of non-edible herbaceous plants (e.g., common grass and water hyacinth, Eichhornia crassipes; https://www.sciencedirect.com/science/article/abs/pii/026974839090130K);
The photosynthetic tissues (e.g., leaves) of edible or non-edible perennial plants;
Legume seeds (including the ‘solubilized’ storage proteins; e.g., soybean, pea, lupin, chickpea);
Macroalgae: (e.g., Oedogonium spp.);
Microalgae: (e.g., Chlorella spp.);
Cyanobacteria: (e.g., Spirulina spp.)

In further embodiments, this procedure may be extended to isolate proteins from animal or other sources.

This application describes, for the first time, a procedure to obtain, at an industrial scale and at a reasonable price, both the total soluble protein and RuBisCO from photosynthetic tissues/cells, as well as the total soluble protein from macro- and microalgae, cyanobacteria and legume seeds in the form of a soluble white powder (or in the form of readily soluble floccules), colourless, tasteless and odourless, and free from toxic metabolites and traces of unhealthy compounds such as organic solvents, heavy metals and other free elements, ions, pesticides, antibiotics, hormones, etc.

The potential applications are endless.

The procedures developed apply to all plant photosynthetic tissues (leaves or not, from herbaceous or perennial, edible or non-edible, land or aquatic plants) and photosynthetic cells, as well as legume seeds and other seeds.

The worldwide demand for protein destined to human consumption has been increasing gradually but permanently for many years and everything points that it will continue so in the future. It seems therefore imperative to find a primary source of protein capable of being produced in an environmentally sustainable way, of undergoing industrial scale production and at a reasonable price.

A plurality of methodologies have been developed in this application which are suitable to undergo scaling-up to an industrial level and at a reasonable price of isolated RuBisCO and of isolated total soluble protein from leafy vegetables, macro and microalgae, cyanobacteria, and by-products of the agricultural and food industries (e.g., leaves from sugar beet), as well as the total soluble legume seed (and other seeds) protein to obtain a plant-derived, colourless, tasteless and odourless, soluble white powder (or readily soluble floccules) free from traces of unhealthy compounds such as organic solvents, heavy metals and other free elements, ions, pesticides, antibiotics, hormones, etc., as well as from those compounds that give a bad taste from the proteins isolated by traditional methods (e.g., from legume seeds, microalgae and cyanobacteria), using a fully sustainable procedure in terms of environment and which may be regarded as a breakthrough in the supply of protein to the world.

At least two methodologies comprise a high-yield and high-efficiency procedure based on the combined use of (i) exquisite precision, differential application of activated charcoal to differentially adsorb distinct protein fractions, pigments and the metabolites responsible for unpleasant taste and (ii) micro-, ultra- and nanofiltration.

Further procedures, directed to isolate RuBisCO, may involve the additional use of calcium phytate and differ by using or not a final step of ethanol precipitation. A further procedure to isolate the soluble proteins from legume seeds, which requires the previous solubilization of the storage proteins which are mostly globulins, as well as the removal of the bad-taste-conferring metabolites. These methodologies may be extended to isolate proteins from animal or other sources.

With the present application, a plurality of different solutions are provided. For the reasons detailed below, both pure RuBisCO and the total soluble protein isolated from edible leaf vegetables, macroalgae and microalgae, cyanobacteria, and by-products of agriculture and the food industries (e.g., leaves from sugar beet) by the proposed methodologies fulfil all requisites highlighted in the above description. So does the isolation the soluble proteins from legume seeds, which requires the previous solubilization of the storage proteins which are mostly globulins, as well as the removal of the bad-taste-conferring metabolites. These methodologies may also be extended to isolate proteins from animal or other sources. In fact, such procedures are so general in what proteomics is concerned that they apply to most protein-containing materials.

The reasoning behind a plurality of the new methodologies, which apply to both pure RuBisCO and the total soluble protein from edible photosynthetic cells/leaves, is based on the differential use of activated charcoal concentrations to control the binding of (i) coloured pigments and many metabolites, elements and ions, (ii) of proteins in general except RuBisCO, and (iii) of all proteins present. The combined additional controlled use of micro-, ultra and nanofiltration will remove larger particles/aggregates, as well as the remaining low molecular mass molecules, elements and ions.

Therefore, under appropriate conditions, a procedure was developed capable of using water to extract the soluble contents of edible photosynthetic cells/leaves, followed by the differential use of activated charcoal to adsorb:
The coloured pigments (e.g., chlorophylls and carotenoids), as well as many metabolites and macromolecules (including numerous undesirable compounds, elements and ions, as well as metabolites responsible for unpleasant taste), leaving in solution the total soluble protein and low molecular mass compounds, elements, and ions.
The coloured pigments and many metabolites and macromolecules, including almost all proteins, leaving essentially RuBisCO in solution.

In addition, the combined use of centrifugation, microfiltration, ultrafiltration and nanofiltration will completely remove larger particles/aggregates, as well as activated charcoal, the remaining molecular mass compounds/elements/ions, including most of the water, leaving behind the isolated total soluble protein or pure RuBisCO dissolved in water in a concentrated form.

A final optional drying step (such as freeze-drying or spray-drying) will yield a white soluble, odourless and tasteless powder (or readily soluble floccules) comprising the total soluble protein or pure RuBisCO.

In the case of legume seed storage proteins, which are globulins and therefore insoluble in water, an additional, previous ‘solubilizing’ step is optionally envisaged.

Other methodologies comprise a more complex procedure involving specific RuBisCO precipitation with calcium phytate, solubilization of the pelleted protein at high pH and removal of pigments by a suitable concentration of activated charcoal, (iii) finalized or (iv) not by ethanol precipitation to yield highly pure RuBisCO.

### BRIEF DESCRIPTION OF THE FIGURES

FIG. 1 shows the electrophoretic polypeptide profile of the total soluble protein (extracted with water) from spinach leaves before and after treatment with 1.9% (w/v) of activated charcoal (standard concentration, 15 min of incubation). The total gel polypeptides were stained with Coomassie Brilliant Blue G-250. The gel was loaded with 10 μL of each sample. M: Molecular masses of standards are indicated on the left and expressed in kDa.

FIG. 2 shows the electrophoretic polypeptide profile of the total soluble protein (extracted with water) from spinach leaves before and after treatment with 1.9% (w/v) of activated charcoal (standard concentration, 1 h of incubation). The total gel polypeptides were stained with Coomassie Brilliant Blue G-250. The gel was loaded with 250 μL of each sample. M: Molecular masses of standards are indicated on the left and expressed in kDa.

FIG. 3 shows electrophoretic polypeptide profile of the total soluble protein (extracted with water) from spinach leaves before and after treatment with 1.9% (w/v) of activated charcoal (standard concentration, 1 h of incubation), with 3.8% (w/v) of activated charcoal (2×standard concentration, 1 h of incubation), or with 0.95% (w/v) of activated charcoal (½×standard concentration, 1 h of incubation). The total gel polypeptides were stained with Coomassie Brilliant Blue G-250. The gel was loaded with 200 μL of the total soluble protein extract (before treatment with activated charcoal) or with 250 μL of the other samples. M: Molecular masses of standards are indicated on the left and expressed in kDa.

FIG. 4 shows the electrophoretic polypeptide profile of the total soluble protein (extracted with water) from spinach leaves before and after treatment with 0.475% (w/v) of activated charcoal (¼×standard concentration, 1 h of incubation) or with 0.317% (w/v) of activated charcoal (⅙×standard concentration, 1 h of incubation). The total gel polypeptides were stained with Coomassie Brilliant Blue G-250. The gel was loaded with 200 μL of the total soluble protein extract (before treatment with activated charcoal) or with 250 μL of the other samples. M: Molecular masses of standards are indicated on the left and expressed in kDa.

FIG. 5 shows the electrophoretic polypeptide profile of the total soluble protein (extracted with water) from spinach leaves after treatment with 1.52% (w/v) of activated charcoal (0.8×standard concentration, 4 h of incubation), with 1.71% (w/v) of activated charcoal (0.9×standard concentration, 4 h of incubation), with 2.09% (w/v) of activated charcoal (1.1×standard concentration, 4 h of incubation) or with 2.28% (w/v) of activated charcoal (1.2×standard concentration, 4 h of incubation). The total gel polypeptides were stained with Coomassie Brilliant Blue G-250. The gel was loaded with 250 μL of each sample. M: Molecular masses of standards are indicated on the left and expressed in kDa. Colour of the solutions (−20%, −10%, +10%, +20%): Colourless.

FIG. 6 shows the electrophoretic polypeptide profile of the total soluble protein (extracted with water) from spinach leaves after incubation with 1.9% (w/v) of activated charcoal (standard concentration) during several periods of time (15 min, 30 min, 1 h, 2 h, 4 h and overnight). The total gel polypeptides were stained with Coomassie Brilliant Blue G-250. The gel was loaded with 250 μL of each sample. M: Molecular masses of standards are indicated on the left and expressed in kDa. Colour of the solutions: “Greenish”−15 min and 30 min; Colourless −1 h, 2 h, 4 h and overnight.

FIG. 7 shows the electrophoretic polypeptide profile of the total soluble protein (extracted with water) from spinach leaves after incubation with 0.95% (w/v) of activated charcoal (½×standard concentration) during several periods of time (1 h, 2 h, 4 h and overnight). The total gel polypeptides were stained with Coomassie Brilliant Blue G-250. The gel was loaded with 250 μL of each sample. M: Molecular masses of standards are indicated on the left and expressed in kDa. Colour of the solutions (1 h, 2 h, 4 h and overnight): Colourless.

FIG. 8 shows the electrophoretic polypeptide profile of the total soluble protein (extracted with water) from spinach leaves after incubation with 0.475% (w/v) of activated charcoal (¼×standard concentration) during several periods of time (1 h, 2 h, 4 h and overnight). The total gel polypeptides were stained with Coomassie Brilliant Blue G-250. The gel was loaded with 250 μL of each sample. M: Molecular masses of standards are indicated on the left and expressed in kDa. Colour of the solutions (1 h, 2 h, 4 h and overnight): Colourless.

FIG. 9 shows the electrophoretic polypeptide profile of the total soluble protein (extracted with water) from spinach leaves after treatment with 0.475% (w/v) of activated charcoal (¼×standard concentration, 1 h of incubation). The total gel polypeptides were stained with Coomassie Brilliant Blue G-250. The gel was loaded with 100 μL of sample. M: Molecular masses of standards are indicated on the left and expressed in kDa.

FIG. 10 shows the total soluble protein (extracted with water) from spinach leaves after treatment with 0.475% (w/v) of activated charcoal (¼×standard concentration, 1 h of incubation), followed by lyophilization.

FIG. 11 shows the total soluble protein (extracted with water) from spinach leaves was treated with 1.9% (w/v) of activated charcoal (standard concentration, 4 h of incubation), followed by lyophilization, leaving isolated RuBisCO (see FIGS. 1, 2 and 6).

FIG. 12 shows the one-step isolation of the total soluble protein (extracted with water) from Spirulina after incubation with activated charcoal (half of the standard concentration, incubation of 2 h) to obtain a soluble powder with no odour or taste. Organolpetic properties of the powder: colour, green; no smell; no taste.

FIG. 13 shows the one-step isolation of the total soluble protein (extracted with water) from Chlorella after incubation with activated charcoal (half of the standard concentration, incubation of 2 h) to obtain a soluble powder with no colour, odour or taste. Organolpetic properties of the powder: colour, “yellowish”; no smell; no taste.

FIG. 14 shows a process diagram for obtaining pure RubisCO.

FIG. 15 shows a further process diagram for obtaining pure total soluble protein.

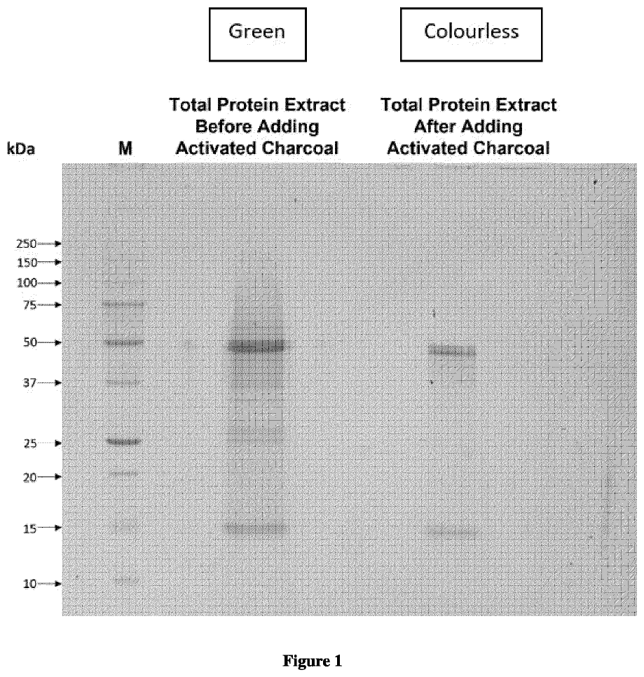

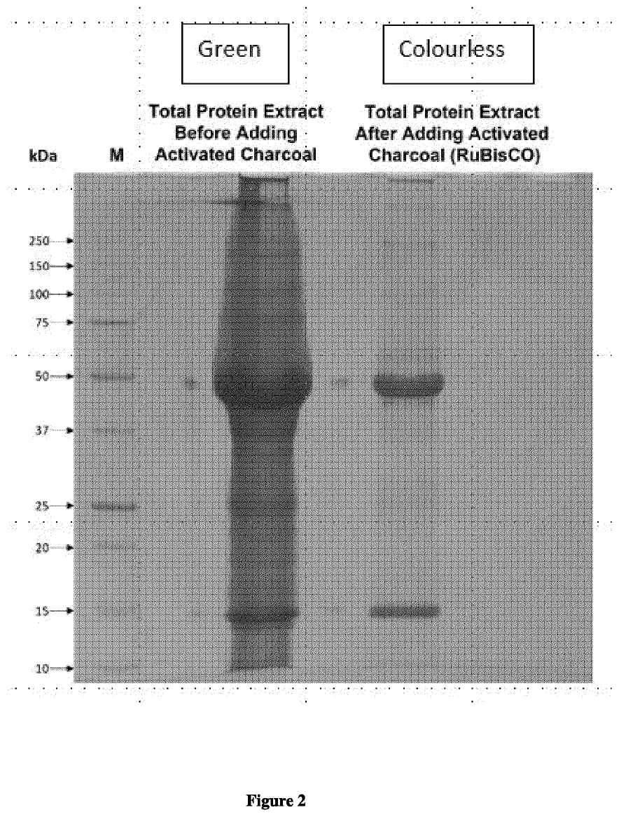

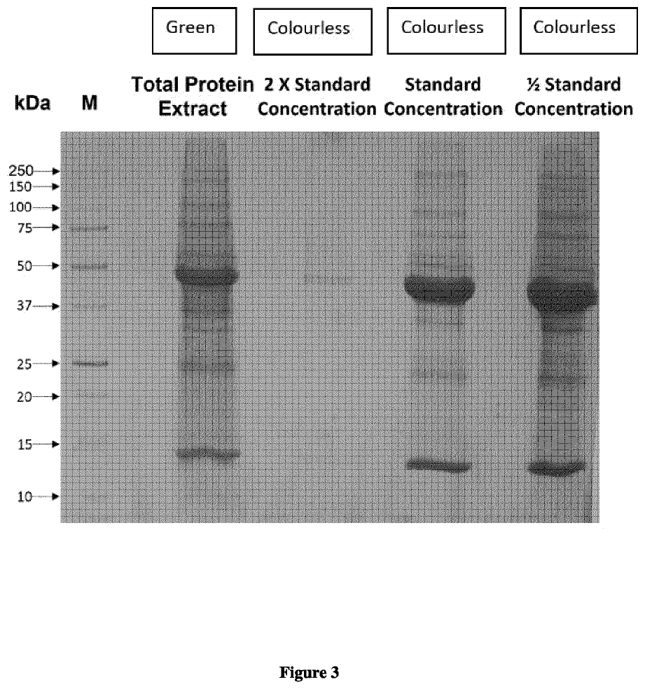

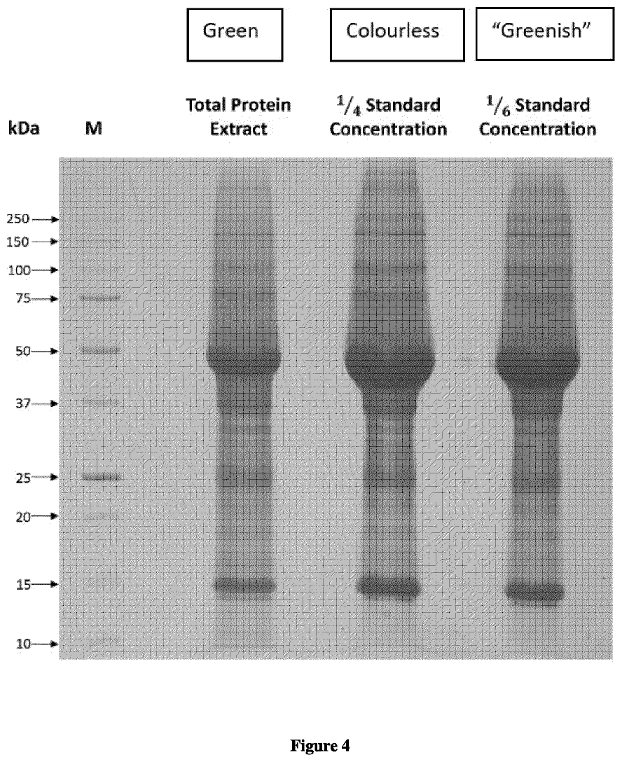

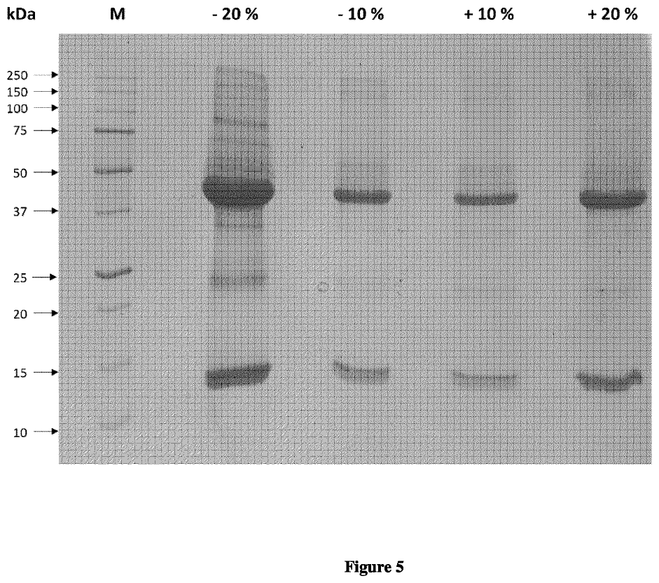

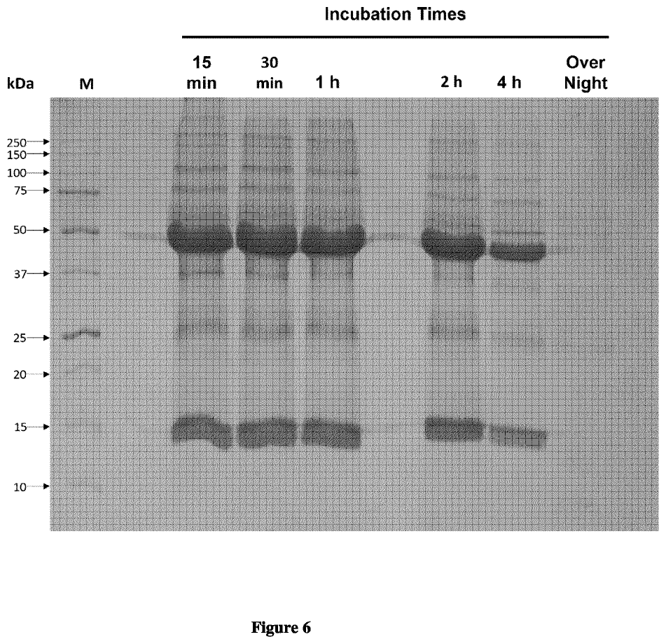

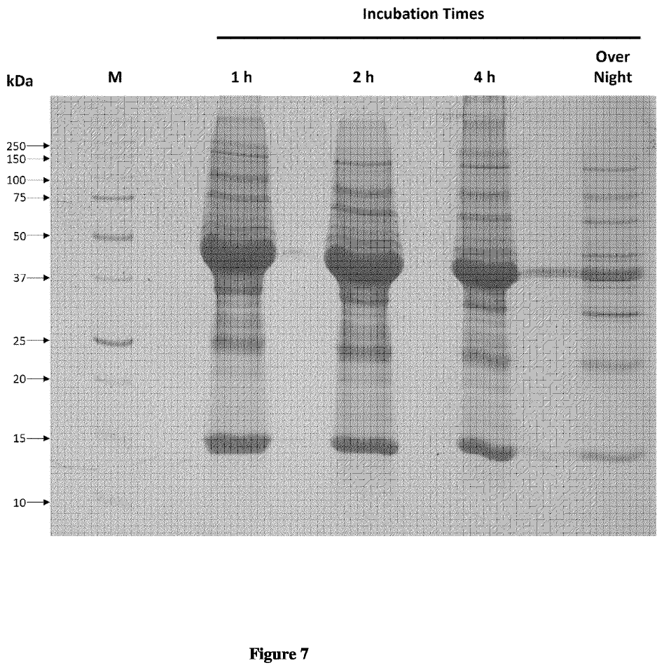

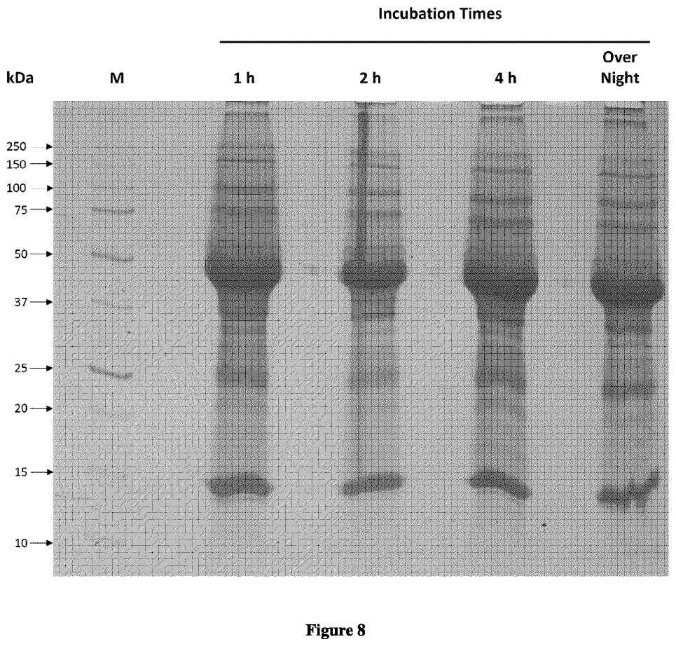

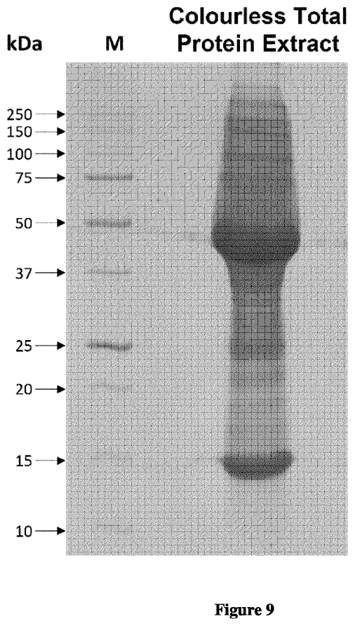

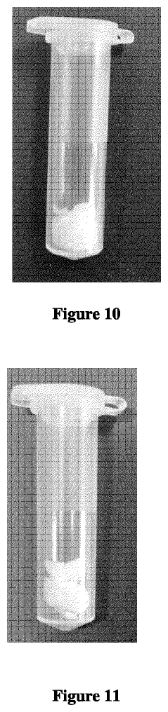

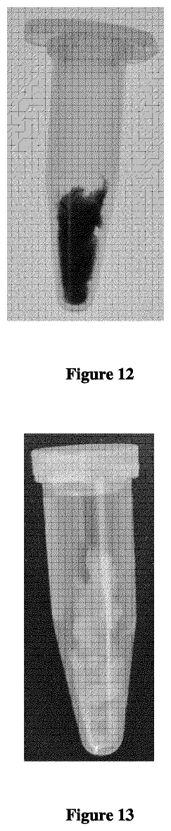

### DETAILED DESCRIPTION OF THE FIGURES

### Specific Embodiments

### Method to Obtain Colourless RuBisCO

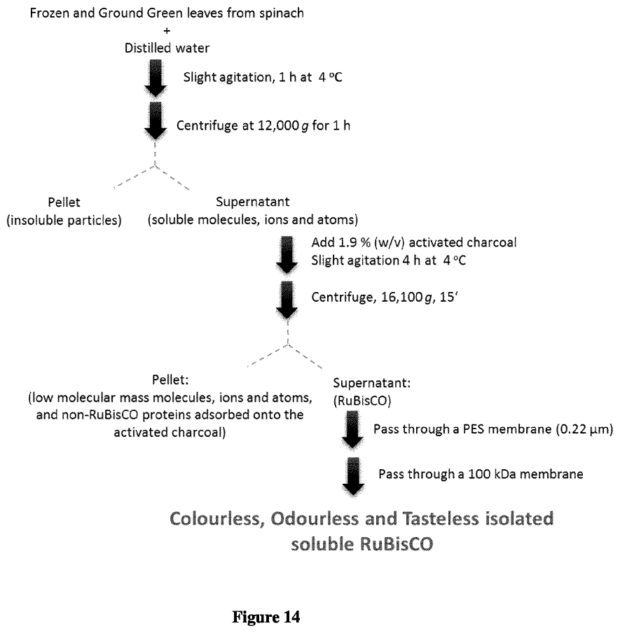

As shown in FIG. 14, green leaves from spinach (Spinacea oleracea; or any other photosynthetic tissue/cell) were frozen and ground to a fine powder followed by the addition of distilled water (1:10 g/mL). The aqueous solution was slightly agitated (1 h, 4° C.) and then centrifuged for 1 h at 12,000 g and 4° C. The pellet was discarded and 1.9% (w/v) of activated charcoal was added to the supernatant (total soluble leaf protein water extract) to remove the green color and the non-RuBisCO proteins, to purify RuBisCO. The supernatant plus the activated charcoal were slightly agitated (4 h, 4° C.) and then centrifuged at 16,100 g for 15 min and 4° C. The pellet (activated charcoal) was discarded, and the supernatant (pure RuBisCO) was filtered through a PES membrane (0.22 μm) to remove all the remains of the activated charcoal. The final step was to subject the colourless RuBisCO water solution to a filtration step through a 100 kDa MWCO membrane to concentrate it and to remove the salts and other low molecular mass compounds still present in the solution.

Colourless, odourless and tasteless pure and soluble RuBisCO is then obtained as a white powder (or readily soluble floccules) following a suitable drying method, such as freeze-drying or spray-drying.

Alternative steps may be implemented and the order of these steps may be varied.

For example, several known procedures may be followed for the aqueous protein extraction from the intact biological material. Furthermore, ethanol may be employed at the precipitation step. In a further embodiment, a high salt concentration may be employed for the pelleted protein solubilization. In further embodiments, dialysis or gel filtration may be employed in the desalting step. In further embodiments, bentonite may be employed at the pigment removal step. Freeze-drying or spray-drying may be undertaken to obtain the protein in a powder form. No alternative steps using harmful substances (e.g., organic solvents) are envisaged.

### Method to Obtain the Colourless Total Soluble Leaf Protein

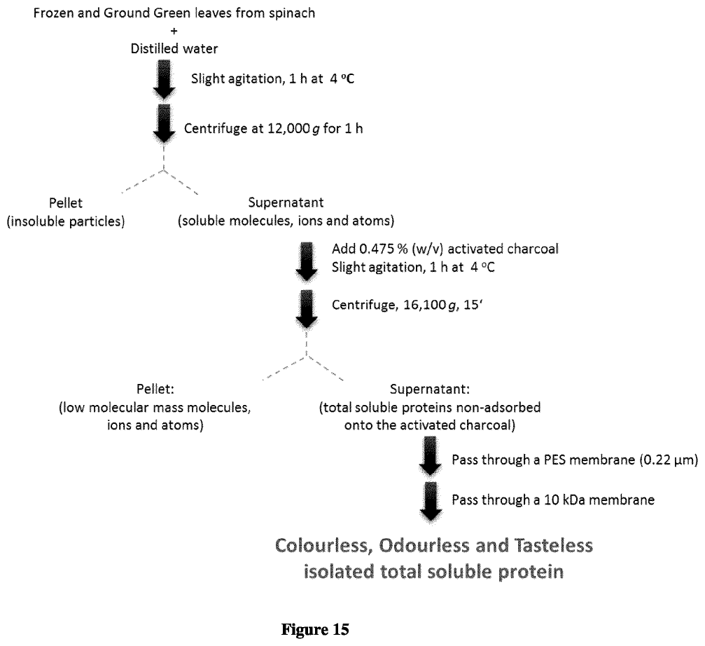

As shown in FIG. 15, green leaves from spinach (Spinacea oleracea; or any other photosynthetic tissue/cell) were frozen and ground to a fine powder followed by the addition of distilled water (1:10 g/mL). The aqueous solution was slight agitated (1 h, 4° C.) and then centrifuged for 1 h at 12,000 g and 4° C. The pellet was discarded and 0.475% (w/v) of activated charcoal (one quarter of the standard concentration) was added to the supernatant (total soluble leaf protein water extract) to remove essentially the green colour (pigments including chlorophylls and carotenoids, as well as many elements, ions and small molecular mass compounds). The supernatant plus the activated charcoal were slight agitated (1 h, 4° C.) and then centrifuged at 16,100 g for 15 min and 4° C. The pellet (activated charcoal) was discarded, and the supernatant (pure total soluble leaf protein, including RuBisCO) was filtered through a PES membrane (0.22 μm) to remove all the remains of the activated charcoal. The final step was to subject the colourless total soluble leaf protein water extract to a filtration step through a 10 kDa MWCO membrane to concentrate it and remove the salts and other low molecular mass compounds still present in the solution.

Colourless, odourless and tasteless pure and soluble total leaf protein is then obtained as a white powder (or readily soluble floccules) following a suitable drying method, such as freeze-drying or spray-drying.

In most embodiments, all protein powders (or readily soluble floccules) are readily soluble in water and free from chlorophyll pigments with the only exception of certain cyanobacteria in which a small proportion of the initially present pigments is retained in the final protein preparations.

### Further Aspects

Concerning the isolation of the typically insoluble legume seed proteins as a colourless, tasteless and odourless, soluble white powder (or readily soluble floccules), the process of solubilization involved preferably a single step to obtain the proteins from legume seeds whilst also removing the unpleasant taste.

Protein content (mg protein/g of seed)

| Extraction procedure | Pea | Soybean | Lupin |
|---|---|---|---|
| Total albumins | 26.4 | 75.7 | 72.4 |
| (standard procedure) | | | |
| Total globulins | 14.8 | 66.7 | 44.8 |
| (standard procedure) | | | |
| NaCl (5% w/v) | 113.5 | 143.7 | 218.1 |
| Bentonite (½ standard | 73.1 | 60.8 | 189.4 |
| dose) | | | |
| Activated charcoal (½ | 62.1 | 81.7 | 164.0 |
| standard dose) | | | |
| Phytate (7.5 mM) | 58.6 | 87.2 | 131.5 |
| Phosphate (100 mM) | 43.6 | 47.9 | 80.6 |

Extraction procedures: in one embodiment, the dry seeds were milled to a fine powder and the total albumins and total globulins extracted following standard procedures. In the remaining procedures, the total proteins were extracted from the flour in water containing one or a combination of NaCl (5% w/v), bentonite (½ standard dose), activated charcoal (½ standard dose), phytate (7.5 mM) and phosphate (100 mM), respectively.

Optionally, the homogenates were centrifuged and the supernatants, containing the soluble proteins, were desalted twice into water, to yields clear solutions comprising the proteins dissolved in water.

To further assess the solubility of the total protein fractions obtained, the above solutions were freeze-dried and the resulting powder freely redissolved in water.

The process has yielded advantageous results for a least the following:
10 to 40 mM EDTA
2.5% to 15%, preferably 5 to 15% (w/v) NaCl
Three bentonite concentrations
Three activated charcoal concentrations
7.5 to 30 mM phytate
25 to 100 mM phosphate

After directly extracting the proteins from the seed flour with each one of these solutions, the solutions were centrifuged and the solubilized proteins separated from the low molecular mass compounds, elements and ions by gel filtration or nanofiltration.

The protein was quantified by the Lowry method, as indicated in the table. When the resulting solution was totally clear (indication full protein solubilization), the abbreviation TS (meaning totally soluble) was placed in the table.

These results were obtained for pea, soybean and lupin.

When compared to the standard extraction of albumins first, then globulins (the first two rows of the Table), all these procedures allow the extraction of a much higher proportion of the proteins present in the seeds.

All methods presented in the Table allow (to different extents) the full solubilization of a large proportion of the seed proteins, all much higher than the standard procedures.

With embodiments of some of the preceding procedures concerning the improved extraction of the proteins from legume seeds, certain embodiments yield one or more of the following:
The seed protein in a soluble form. The amount of soluble-in-water protein obtained is greater than that corresponding to the water-soluble albumins plus the water-insoluble globulins.
In a preferred embodiment of the procedure, the isolated protein solubilized in water is obtained, then dried to a powder and then, when required, solubilized again in water to yield a clear solution.
The solubilized protein is, in preferred embodiments, a tasteless and odourless form. In the case of soybean, for example, the unpleasant taste is removed.
Preferably, low molecular mass natural compounds are removed (flavonoids with estrogenic activity in soybean, alkaloid residues in sweet lupin, etc.).
Preferably, if present in the initial biological material, residues of pesticides, antibiotics, hormones, heavy metals are removed.
In preferred embodiments, no toxic ingredients (i.e., organic solvents) or any other ingredients (except those mentioned in the patent application) are used.

Combination of certain methods are envisaged in the claims which now follow.

1. A process of obtaining a RuBisCO preparation from photosynthetic material, comprising the steps of:
lysing said photosynthetic material to extract RuBisCO accompanied by a fraction of the chlorophylls of said photosynthetic material; and
submitting at least a portion of said extracted material to an activated charcoal; whereby a further portion is removed by or adsorbed onto said activated charcoal.

2. The process of claim 1, wherein said further portion comprises chlorophyll pigments.

3. The process of claim 1, comprising a step of grinding leaves containing photosynthetic material and adding water to obtain an aqueous solution.

4. The process of claim 3, comprising the step of agitating and/or centrifuging said aqueous solution in order to obtain a pellet of insoluble particles and a supernatant.

5. The process of claim 4, comprising the step of adding said activated charcoal to said supernatant and further processing the solution whereby said activated charcoal adsorbs chlorophyll pigments.

6. (canceled)

7. The process of claim 5, wherein said further processing results in a pellet of low molecular mass molecules, ions and/or atoms and a supernatant of soluble proteins which were non-adsorbed onto said activated charcoal.

8. The process of claim 1, wherein the process further comprises a step of centrifugation to separate out pellets and a supernatant comprising the soluble proteins which were not adsorbed onto the activated charcoal.

9. The process of claim 1, which further comprises one or more further steps of nanofiltration, ultrafiltration or microfiltration; whereby soluble RuBisCO is isolated.

10. The process according to claim 9, wherein said nano-filtration employs a PES (polyethersulfone) membrane of for example 0.22 μm.

11. The process according to claim 9, wherein said nano-filtration employs with a molecular weight cut-off membrane of 100 kDa or less.

12. (canceled)

13. A RubisCO preparation obtained by a process according to claim 1.

14. A process of obtaining a protein preparation from biological tissues selected from the group comprising seeds, algae, cyanobacteria and animal products, said process comprising the steps of:
lysing said biological tissues to extract protein; and
submitting at least a portion of said extracted material to an activated charcoal; whereby a portion is removed by or adsorbed onto said activated charcoal; and/or submitting a portion of extracted material to a solution containing EDTA eg. of 10 to 40 mM; and/or submitting a portion of extracted material to a solution containing NaCl eg. of 2.5 to 15% (w/v); and/or submitting a portion of extracted material to a bentonite concentration; and/or submitting a portion of extracted material to a solution containing phytate eg. of 7.5 to 30 mM; and/or submitting a portion of extracted material to a phosphate eg. of 25 to 100 mM.

15. The process of claim 14, wherein said further portion comprises chlorophyll pigments.

16. The process of claim 14, comprising a step of grinding biological tissues and adding water to obtain an aqueous solution.

17. The process of claim 16, comprising the step of agitating and/or centrifuging said aqueous solution in order to obtain a pellet of insoluble particles and a supernatant and further comprising the step of adding said activated charcoal to said supernatant and further processing the solution whereby said activated charcoal adsorbs a further portion.

18. (canceled)

19. (canceled)

20. The process of claim 17, resulting in a pellet of low molecular mass molecules, ions and/or atoms and a supernatant of soluble proteins which were non-adsorbed onto said activated charcoal.

21. The process of claim 20 wherein the process further comprises a step of centrifugation to separate out pellets and a supernatant comprising the soluble proteins which were not adsorbed onto the activated charcoal.

22. The process according to claim 14, which further comprises one or more further steps of nanofiltration, ultrafiltration or microfiltration; whereby one or more of the following proteins are isolated: legume seed proteins, RuBisCO, soluble protein from leafy vegetables either edible or non-edible, macro or micro algae protein, cyanobacteria protein, animal products protein.

23. The process according to claim 22, wherein said nano-filtration employs a PES (polyethersulfone) membrane of for example 0.22 μm.

24. The process according to claim 22, wherein said nano-filtration employs an ultra-filtration membrane with a molecular weight cut-off membrane of 100 kDa or less.

25. (canceled)

26. (canceled)
